(19) United States
(12) Patent Application Publication
(10) Pub. No.: US 2007/0060875 A1
(43) Pub. Date: Mar. 15, 2007

(54) DUAL CHAMBER CONTAINER WITHOUT BY-PASS IN THE CYLINDRICAL BODY

(76) Inventors: Stefan Bassarab, Biberach (DE); Alexander Bauer, Biberach (DE); Nicole Denkinger, Biberach (DE); Patrick Garidel, Norderstedt (DE); Markus Hemminger, Biberach (DE); Hans-Joachim Kern, Mittelbiberach (DE); Andreas Langer, Maselheim (DE); Ingo Presser, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/464,491

(22) Filed: Aug. 14, 2006

(30) Foreign Application Priority Data
Aug. 13, 2005 (DE) 10 2005 038 458

(51) Int. Cl. A61M 37/00

(52) U.S. Cl. 604/84; 604/85

(57) ABSTRACT

The invention relates to a double chamber container for holding and combining two separate components, at least one of which is liquid, comprising a cylindrical body (15) with a closure (20, 60) at each end (5, 70), of which the closure (60) at the end of a chamber containing the liquid component (50) can be pushed, and a separating stopper (40) which can only be pushed by increased pressure, which is mounted in the cylindrical body (15), which defines the volume of the chamber (30, 50) mounted above and below, as a seal between the two chambers (30, 50), with an opening (42) running therethrough along its longitudinal axis, said opening having an upper part (42a) to the side of the chamber containing the liquid component (50) that is closed off in fluid-tight manner by a shaped member that is movable by gentle to moderate pressure (45).

This application claims benefit under 119(a) of DE 10 2005 038 458, which was filed on Aug. 13, 2005, and which is incorporated herein by reference in its entirety.

### BACKGROUND OF THE INVENTION

1. Field of the Invention

The present invention relates to a double chamber container without a bypass in the cylindrical body, a method of filling it and the use thereof.

2. Description of the Prior Art

There are pharmaceutical compositions which in their ready-to-use form very rapidly lose their efficacy. To enable these compositions to be used in spite of their short shelf life, two-compartment systems have been developed, for mixing them immediately before use.

Thus, pharmaceutical preparations which cannot be used in solution over long periods may be made durable by lyophilization, for example, and possibly stored away from light. The dry substance is only dissolved again, i.e. reconstituted, immediately before use.

As is known, lyophilizates are also used in ready-prepared syringes with two-chamber systems—the lyophilizate and solvent being stored separately and only combined just before use. Two-chamber prepared syringes of this kind have already been described in “Die Lyophilisierung von Arzneimitteln in Fertigspritzen”, H. Vetter, Die Pharmazeutische Industrie, 46th year, 1984, No. 10, p. 1045-1049. These ready-prepared syringes were developed in order to do away with the usual major effort of handling two sterile sealed containers and a syringe and to rule out the risk of confusion, for example, with unlabelled disposable syringes.

The ready-prepared syringes are usually cleaned, siliconized, and provided with a guard for the cannula. The siliconization for coating the primary packaging means is carried out so that the rubber stopper can easily be pushed into the glass cylinder. Both the rubber stopper and the inside of the glass cylinder are covered with a silicon oil. Then the ready-prepared syringes can be pre-sterilized in batches and filled under sterile conditions. They are then fitted with a cylinder stopper and finished, at which time the plunger rods are fitted. Then they are labeled and packaged. The filling and sealing are carried out from the rear end of the syringe, as the opening at the cannula end is too small.

Specifically, ready-prepared syringes of this kind are constructed so that each of the components is housed in its own chamber, the chambers being arranged axially behind one another and only brought into contact with one another immediately before use, so that the liquid component in one chamber can flow over to the dry component in the other chamber. This produces the desired mixture. It is known to provide a bypass for this purpose, for example in the form of a widening in the internal diameter of the glass cylinder in the syringe, through which the liquid component can flow when a stopper separating the two chambers has been pushed so far into the bypass that the liquid component can flow around the stopper.

In the prior art attempts were made to find improved solutions enabling the two-component systems to be mixed while maintaining the sterility of the two components. There are numerous proposals along these lines, while in the present case only those systems which do not have a bypass as described above are of interest.

The patents described in the following paragraphs, namely DE 32 13 072, DE 1 809 892, U.S. Pat. Nos. 5,785,683, 6,602,223, EP 1 038 543, and DE 25 46 495, are incorporated herein by reference in their entireties.

For example, DE 32 13 072 describes a syringe which is subdivided into two compartments for holding a powdered medicament and its solvent, the mixing being carried out by withdrawing the plunger rod of the syringe, so that the solvent can flow around the plunger.

In DE 1 809 892 a two-chamber syringe is described, the filling of which is made easier by the fact that the liquid is added through a filling channel provided in the plunger rod. In U.S. Pat. Nos. 5,785,683 and 6,602,223 the process of mixing the two components in the syringe is achieved using specially designed valves which can be controlled by moving the plunger.

Moreover, EP 1 038 543 discloses a twin-chamber syringe for medical purposes which contains a plunger rod with projecting latching elements which are arranged in diametric pairs and/or at a rotation angle of 90° to ensure a controlled mixing process and sequential administration of the mixture.

It is also known from DE 25 46 495 to connect a syringe via a special stopper to a container which is sealed off by a second stopper and expelled before the mixing process.

All the systems described have the disadvantage of being very complex in construction, based on specially designed valves or plunger rods. DE 25 46 495 in particular has, in addition to a complex structure which connects a syringe and a container via a connecting member, the serious disadvantage that the stopper drops into the container and comes into contact for quite a lengthy period not only with the solvent injected in but also with the solid contained therein. In addition, this stopper may cause problems during the withdrawal of the mixture as it may block the withdrawal opening and may possibly hold back or absorb some of the mixture.

Another disadvantage is that 2 containers have to be joined together, the actual weak point being the connecting member, which may slip or fall off during lengthy storage. Therefore there is the risk of contamination from outside. Moreover, this design is very bulky and therefore not very easy to store and handle.

There is therefore still a need for a device which is easy to handle, for the reliable storage and mixing of two-component systems, at least one of which is in liquid form. At the same time, contamination from outside should be prevented as far as possible, i.e. the mixing should take place without removing the components from the sterile interior of the device and without compromising the sterility. The device should also be easy to store. In addition, an easy method of filling such a device should be provided. The device and the method should also be suitable for implementation on an industrial scale.

### SUMMARY OF THE INVENTION

The objective described above is achieved by the features of claim 1.

According to this, a double chamber container for holding and combining two separate components, at least one of which is liquid, is provided, comprising a cylindrical body with a closure at each of the two ends of the body, of which the closure at the end of the chamber containing the liquid component can be pushed by the application of pressure, and

a separating stopper which can only be pushed along by the application of greater pressure, which is mounted in the cylindrical body, which defines the volume of the chamber mounted above and below as a seal between the two chambers, with an opening running right through the separating stopper along its longitudinal axis, said opening having an upper part and a lower part,

wherein the upper part of the opening to the side of the chamber containing the liquid component is closed off in fluid-tight manner by a shaped member which is movable by the application of gentle to moderate pressure.

### BRIEF DESCRIPTION OF THE FIGURES

The accompanying Figures illustrate the device according to the invention and the procedural teaching which is to be carried out according to the invention without restricting the invention thereto. Specifically:

FIG. 1 shows an embodiment of the invention of a container according to the invention in the filled state with a closed bypass in the separating stopper;

FIG. 2 shows an embodiment of the invention of a container according to the invention with an open bypass in the separating stopper;

FIG. 3 is a detailed view of a separating stopper according to the invention;

FIG. 4 is a plan view of an embodiment of a separating stopper according to the invention and

FIG. 5 shows various embodiments of a shaped member according to the invention.

### LIST OF REFERENCE NUMERALS

5 end on the solid side (side B)
10 container
15 cylindrical body
20 closure, mouth
30 chamber containing solid component
40 separating stopper
42 opening, channel
42a upper part of the opening 42 with the diameter D1
42b lower part of the opening 42 with the diameter D2
45 shaped member
47 bypass
50 chamber containing liquid component
60 closure, stopper
70 end on the liquid side (side A)

### DETAILED DESCRIPTION OF THE INVENTION

The objective described above is achieved by the features of claim 1.

According to this, a double chamber container for holding and combining two separate components, at least one of which is liquid, is provided, comprising

a cylindrical body with a closure at each of the two ends of the body, of which the closure at the end of the chamber containing the liquid component can be pushed by the application of pressure, and

a separating stopper which can only be pushed along by the application of greater pressure, which is mounted in the cylindrical body, which defines the volume of the chamber mounted above and below as a seal between the two chambers, with an opening running right through the separating stopper along its longitudinal axis, said opening having an upper part and a lower part,

wherein the upper part of the opening to the side of the chamber containing the liquid component is closed off in fluid-tight manner by a shaped member which is movable by the application of gentle to moderate pressure.

Accordingly it is as if an internal “bypass” is provided, integrated in the separating stopper between the two chambers of a cylindrical container. The particular construction according to the invention of the separating stopper between the two components in a two-chamber system is such that the function of the bypass usually formed in ready-prepared syringes is integrated therein. This is achieved by means of a through-opening along the longitudinal axis of the separating stopper, the opening being divided into an upper part and a lower part and a shaped member suitably sealing off the upper part of the opening in fluid-tight manner.

During use of the separating stopper constructed according to the invention, with a through-opening which is closed off at the upper part towards the liquid side by a shaped member, the pressure on the liquid side increases as a result of the application of an external force on the liquid, leading the shaped body to be pushed out of its position in the upper part into or through the opening or the opening channel and thereby finally open up the latter. The two components may be mixed in this way without compromising the sterile conditions of the double chamber container.

Accordingly the separating stopper cannot be moved by the gentle to moderate application of pressure”, i.e., it should essentially retain its position in the cylindrical container when force is applied for example using the fingers or a punch on the upper closure and hence on the liquid side, whereas the shaped member should be “displaceable”, i.e. it should leave its position when a force is exerted which is sufficient to move the upper closure, i.e. a gentle to moderate pressure, and hence when corresponding force is applied to the liquid side to enable the two components to mix. The separating stopper can only be moved if “increased pressure” is applied to the closure stopper, e.g., when the upper closure or stopper has penetrated so far into the glass body that it is in contact with the separating stopper and as further force is exerted pushes it into the glass body as well.

Thus, according to the invention one chamber may contain a liquid and the other a solid. The invention is not restricted as to the nature of the two components, provided that at least one component is liquid. For example, a solid powdered pharmaceutical composition, preferably a lyophilizate, i.e. a freeze-dried medicament, may be present. In lyophilization or freeze-drying, a liquid product is frozen and then the frozen product is dried by sublimation. Preferably, the liquid component may be for example a dissolving or dispersing medium for the solid; it may be for example a reconstituting medium for the lyophilizate. If the solid is a pharmaceutical composition, an injectable solution may be prepared, for example, in either dissolved or dispersed form. The solvent is preferably water, but may also be some other solvent or a mixture of solvents.

The cylindrical body according to the invention is an essentially elongate hollow body with two open ends which has preferably been formed integrally, i.e. made in one piece, and subdivided by the separating stopper into chambers. The “cylindrical” body need not necessarily be cylindrical in shape, although this is the most common shape. Any other geometric shape for an elongate hollow body is possible, such as angular or oval, for example, in which case the closures and separating stopper and the like must be matched to the chosen shape. The material of which the cylindrical body consists or which it contains is not particularly restricted according to the invention. The container may be selected for example from plastics or glass. Glass is preferred on account of its transparency and its compatibility with numerous medical formulations. The cylindrical body therefore preferably consists or glass or contains glass, as this produces the least effect on the components contained therein and the body is preferably transparent. For particular requirements, however, other materials may be suitable, such as special plastics or the like. Medical safety is particularly important, as it is desirable that there be as little interaction as possible with the medium contained therein.

Preferably, the cylindrical body has the same diameter over its entire length.

The separating stopper arranged in the cylindrical body defines the size/volume/dimensions of the two chambers and functions together with the shaped member as a liquid barrier device for the liquid component, which is prevented from passing into the other chamber.

As already explained, the separating stopper is arranged virtually “fixedly” in the cylindrical body and should not therefore be movable when a force which is sufficient to move the upper closure is applied to the chamber containing the liquid component. The separating stopper is preferably made of rubber or a rubber-like elastic material such as plastics, particularly natural and synthetic rubber, thermoplastics, elastomers, thermoplastic elastomers, and the like. The separating stopper should provide a guaranteed seal between the two chambers and should have the necessary strength to stay in place even when moderate pressure is applied.

According to a preferred embodiment according to the invention the separating stopper is of a suitable shape, size, and/or material that will not prevent it from being pushed out of its fixed and defined position in the cylindrical body as intended. This may conveniently be achieved by the corresponding provision of a suitable shape with projections, e.g., knobs, lips, beads or webs and/or for example by the choice of a diameter which is larger than the internal diameter of the cylindrical body.

In the present invention the term “form” is intended to refer to the outer shape or geometry. The term “size” is intended to refer to the dimensions, i.e. the ratios of magnitude.

If the liquid is put under pressure, the separating stopper continues to adhere to the inner wall of the cylindrical body by frictional forces. If the frictional adhesion of the separating stopper to the wall of the cylindrical body is not sufficient for a particular application, to prevent accidental movement, the latter may be provided with small suitable projections and/or for example secured by means of sealing lips, beads, or surfaces to adhere to the inner wall of the cylindrical body. The pressure therefore does not increase in the other chamber. As a result, a differential pressure is produced between the two chambers, as a result of which the shaped member is moved from the upper part of the opening towards the other chamber to the lower part of the opening. As soon as the shaped member leaves the opening free, the solvent is able to flow through the opening from one chamber into the other.

The shape of the shaped member is not particularly restricted. It has a suitable three-dimensional form to ensure that the opening in the upper part is closed off. The shaped member closes off at least the upper part of the opening on the side nearest the chamber containing the liquid component. An “upper part” within the scope of the present invention means a part of the opening as a whole with a defined length L1, while the opening of the separating stopper is not generally closed over the entire length by the shaped member but only a part of a predetermined length (upper part) defined by the chosen dimensions of the shaped member and/or the opening, adjacent to which is another defined portion of the opening (lower part) into or through which the shaped member is moved by the application of the external pressure.

The upper and lower parts may have the same shape and size, so that the shaped member does not free the opening until it has left both portions.

The shaped member preferably has an outer diameter which is greater than the inner diameter of the opening in the upper part, so that sufficient pressure is built up between the inner wall and shaped member to close off the interface but so that the shaped member—unlike the separating stopper—is movable in the container under the effect of the force applied for the mixing process.

Examples of suitable three-dimensional shapes for the shaped member are round, oval, cylindrical shapes, cylindrical shapes with rounded sides, dumbbell-shaped, cuboid, conical, truncated frustum or conical shapes.

Thus, according to the invention, a suitable shape and/or size for the opening in the separating stopper and a suitable shape and/or size for the shaped member are selected so that when pressure is applied the shaped member frees the opening far enough to allow the liquid component to flow into the chamber containing the solid component.

The opening need not be round like a bore, but may also be oval, polygonal of some kind, e.g., square or rectangular, or it may have any desired three-dimensional shape.

Particularly preferably according to the invention the geometric shape and/or size of the opening and shaped member are matched to one another such that the shaped member under the application of pressure can be moved out of its original position in the upper part of the opening in which it constitutes a seal for the liquid component, but cannot entirely leave the opening in the lower part. It is thus preferred if the shaped member does not penetrate into the chamber containing the solid.

This may be achieved for example if the opening in the upper part on the side of the chamber containing the liquid component has a different geometric shape and/or size from that in the lower part on the side of the chamber containing the solid component. Therefore the shape of the opening does not have to be the same over the entire length, and/or the inner diameter may vary after a certain length. Particularly preferably, for example, a suitable tapering at the end of the separating stopper nearest the chamber containing the solid may be used to prevent the shaped member from penetrating into the front chamber containing the solid and possibly prevent the product from being removed altogether.

It is therefore preferable if after allowing the liquid component to gain access to the solid component the shaped member does not leave the access channel or lower part and is secured therein. Suitable configurations may be chosen for the lower part of the opening and the shaped member so that the shaped member cannot leave the opening in the separating stopper but enough room remains for the liquid component to pass into the solid component.

It is particularly preferred according to the invention if the opening in the upper part on the side of the chamber containing the liquid component has a smaller inner diameter D1 than the lower part on the side of the chamber containing the solid component with a larger inner diameter D2. It is particularly expedient if the opening (in the upper part) has a diameter D1 over a length L1, where L1 □ D1 and the opening in the upper part after the length L1 widens out to the diameter D2 over a length L2 (lower part), where: D2>D1 and L2>DK, where DK represents the diameter of the shaped member.

Particularly preferably the opening in the lower part towards the end with the solid comprises means for preventing the shaped member forced out of its original position from leaving the opening. These may be knobs, lips, beads or webs mounted in the lower part of the opening and hence in the liquid channel, which may for example be offset and spaced apart by a defined amount. However, these means are not restricted within the scope of the present invention provided that they prevent the shaped member from leaving the opening (of the lower part) of the separating stopper, but at the same time do not interfere with the passage of the liquid component.

The securing of the shaped member may be achieved not only by means of the dimensions of the opening and shaped member or additional retaining means but also specific geometries may be selected from the opening and shaped member which are deliberately matched to one another. Thus, the opening at one end may be round (upper part) and sealed off with a round shaped member. By the application of pressure the shaped member then moves out of its position and continues to move in the opening, together with the liquid, until it reaches a position, preferably towards the solid-chamber end of the opening, where the opening (lower portion) has a shape through which the shaped member will not fit and is therefore held in place. This may be for example a rectangular, star-shaped or oval form, it may be star-shaped with sharply converging or rounded points or may assume any desired three-dimensional shape which can be closed off by the shaped member at one end (upper part of the opening) and allows liquid to pass through at the other end (lower part of the opening). However, according to this embodiment, because of the geometric conditions selected, liquid can still flow virtually unrestricted into the other chamber, as the shaped member cannot close off the opening completely.

Advantageously, the opening may additionally have one, two or more bypass channels which may be provided in the lower part underneath the shaped member mounted in the separating stopper, in the opening channel before the two components are actually mixed together. They may be one or more recesses in the inner wall in the longitudinal direction, such as a channel or groove. These additionally enable liquid component to flow round the shaped member. This enables the two components to be mixed together particularly rapidly. The mixing may for example be the reconstitution of a lyophilizate. During this step, no other part of the container comes into contact with the liquid or solid component.

The double chamber container further comprises 2 closures, one of which is provided at the solid side And the other at the liquid end. The closure means are not further restricted, provided that the closure device at the liquid end enables pressure to be applied to the liquid so that the shaped member moves out of its position in the separating stopper.

The closures may be selected for example from stoppers, membranes, discs, particularly sealing discs, optionally with flanged caps made of metal and/or plastic caps.

It is particularly advantageous if the closure at the liquid end of the opening is a stopper which may be made of rubber or rubber-like elastic material, such as plastic. The closure at the solid end of the opening may preferably be a pierceable membrane with an outer cap which is flanged, for example, over the outer edge at the emptying end. A removable disc attached to the outer cover may also be provided over it. If the container is a carpule or ampoule, a rubber closure is provided at one or both ends, which can be pierced with an injection needle. The closures may also be removably fixed. In any case, care must be taken to ensure that the closures are sterile.

Preferably the double chamber container is a container for separately storing 2 substances, such as, e.g., a cylinder ampoule or carpule, which is intended for single or multiple use.

The measurements of the double chamber container depend on the volume of the solution which is to be produced; in human medicine, volumes of 10 ml are rarely exceeded, which means that volumes of up to about 20 ml are sufficient. In exceptional cases and for veterinary use however it is possible to exceed these volumes by a long way.

The invention also relates to a method of filling the double chamber container according to the invention, comprising the following steps:
(1) inserting a separating stopper which can only be moved by the application of increased pressure into the cylindrical body with an opening extending right through it along the longitudinal axis of the separating stopper, which has an upper part and a lower part, the upper part of the opening being sealed off in fluid-tight manner by a shaped member which can be moved by slight to moderate pressure;
(2) packing a solid component into side B of the cylindrical body provided with the separating stopper or
(3) adding a liquid containing the solid component into side B of the cylindrical body provided with the separating stopper, then lyophilizing the liquid, to obtain the solid component and sealing this chamber on side B,
(4) adding the liquid component to the cylindrical body through the separating stopper (side A) and
(5) sealing the double chamber container.

The process will hereinafter be described in detail; any individual features described in relation to the process also apply accordingly to the double chamber container and vice versa. In Step (1) a separating stopper which can only be moved by the application of increased pressure is introduced into the cylindrical body having an opening extending right through it along the longitudinal axis of the separating stopper, which has an upper part and a lower part, the upper part of the opening being sealed off in fluid-tight manner by a shaped member which can be moved by slight to moderate pressure. This separating stopper may preferably be placed in the centre of the cylindrical body, but may also be positioned anywhere, depending on the particular application and the desired size of the two chambers.

Then in step (2) or (3) either a solid component or a liquid containing the solid component is packed into side B . In the latter case the cylindrical body is then filled with a lyophilization solution through the open lower end (side B) of the cylindrical body. This may be any desired solution which is to be lyophilized. It may for example be a medicament which is to be made durable by the lyophilization process. The lyophilization is carried out in a manner known in the art, with the solvent escaping through the open lower end of the cylindrical body. The lyophilizer is of a standard commercial kind, the process parameters of which are automatically regulated, for example.

Obviously, this “lower” end faces upwards during the filling process. The terms “top” and “bottom” do not mean that the part of the container thus specified is always at the top or bottom; this is intended primarily as an unambiguous description. For example, the container may be inverted during the lyophilization, but the term originally used is retained for reasons of clarity.

The lower end or the mouth of the cylindrical body is then sealed. The lower end may for example also have a taper. The closure may be for example a stopper, a membrane, a disc, particularly a sealing disc, optionally with a cover, such as a covering cap or flanged cap, or the like. The closure is not particularly restricted as long as it provides a suitable seal, is inert with respect to the medium which is to be added, and meets the conditions of sterility. The lower seal is most particularly preferably a pierceable membrane, a rubber stopper, or an elastic disc, optionally with a flanged cap. It is also possible to provide a removable closure. If the container is a carpule or ampoule, a rubber closure may be provided which can be pierced with an injection needle. However, any other seal known in the art may also be used.

Then the container is inverted and taken to a filling station, where in step (4) it is filled with reconstituting medium through the open top end of the cylindrical body, i.e., the solvent or dispersing medium is poured into the upper chamber (side A) through the separating stopper. After the container has been filled with reconstituting medium, i.e., a dissolving or dispersing medium for the lyophilizate, the container is fitted with a closure (step (5)). Preferably a stopper is used as the upper closure. However, any other closure known in the arm may be used provided that it can be moved by the use of pressure. It is expedient if the upper closure at the liquid end (side A) is a stopper which may be made of plastic or similar materials. Examples of materials are rubber or rubber-like elastic material such as elastomers, thermoplastics, elastomeric thermoplastics, glass, and/or metal, such as stainless steel or similar materials.

It is particularly expedient if the stoppers and/or closure members are supplied and inserted by means of a washing and sterilizing device or an autoclave along sterile corridors. After the container has been sealed it is taken out of the sterile area through an airlock; finally it is labeled and packaged. It will be understood that in this process all the surfaces and equipment are designed for aseptic operation.

The invention also provides a method of mixing 2 separate components, at least one of which is liquid, in a double chamber container, comprising a cylindrical body with a closure at each of the two ends of the body, of which the closure at the end of the chamber containing the liquid component (side B) can be moved by the application of pressure, and

a separating stopper which can only be moved by the application of increased pressure, arranged in the cylindrical body, which defines the volume of the chambers arranged above (side A) and below (side B), as a seal between the two chambers, having an opening running right through it along the longitudinal axis of the separating stopper, which has an upper part and a lower part,

the upper part of the opening at the end of the chamber containing the liquid component being sealed off in fluid-tight manner by a shaped member which can be moved by the application of slight to moderate pressure,

comprising the steps of:

applying pressure to the movable closure;
pressing the shaped member out of its defined position into the upper part of the opening;
transferring the liquid component from one chamber into the chamber containing the solid component to obtain a mixture of both components.

As already described in detail, after being pressed out of its defined position in the upper part of the opening the shaped member is preferably held in a position in the lower part of the opening in which the liquid component is still able to move, as before, from the upper chamber into the chamber containing the solid component

The invention further relates to the use of the container according to the invention in human and veterinary medicine.

The present invention has a number of advantages:

Thus, the internal “bypass” in the separating stopper enables the two component system to be stored without any problems without any premature mixing and hence loss of efficacy of the components. The two-component system provided according to the invention can be stored in its sterilized, pre-filled, ready-to-use state. The two components are mixed together after storage, immediately before use. The double chamber container can be thrown away after use.

A device is provided by means of which the desired solution of the two components is produced immediately before use, resulting not only a fast and reliable system, but also ease of manufacture and filling. The separating stopper allows the volume of the two chambers to be selected at will, so that the container according to the invention can be designed with the proper dimensions for the desired use.

Using the double chamber container according to the invention it is possible to carry out mixing, e.g., reconstitution of a lyophilizate in a sealed two-chamber system, without having to provide the container per se with an additional external or internal bypass and/or without having to break the seal of the system in order to carry out the mixing, thereby opening it up to undesirable external microbial, chemical or physical influences.

Accordingly, there is no risk of contamination by foreign bodies which might penetrate from outside, such as bacteria, particles of dirt, splinters, etc. The container can be used as primary packaging and stored in its clearly labeled form. The use of two-component systems, particularly lyophilized preparations, can thus be made simpler.

There is also the possibility, by suitable dimensioning of the opening in the separating stopper and of the shaped member, to adjust the flow velocity or quantity of liquid flowing through per unit of time accordingly. In this way a correspondingly slow entry or rapid overflowing from one chamber into the other can be controlled by the size of the opening, e.g., in the lower part, in the separating stopper. There is no need to restrict the overflow speed of the liquid component using additional equipment, which would involve considerable expense. The problem whereby in syringes, when two much pressure is applied to the stopper, the liquid component surges forward too quickly over the bypass to the chamber containing the dry substance and as a result any substance which has already been dissolved leaves the cannula of the syringe, does not arise according to the invention, as no syringe is used. Moreover, related undesirable contamination of the user and loss of the dissolved substance and hence a dosage which can no longer be precisely controlled is thus prevented.

A further advantage of the invention is that the double chamber container required for this system has no outer irregularities, in the form of a bypass with one or more protuberances and can therefore be machined more easily and reliably on the usual processing equipment. This has advantages in, for example, filling, lyophilization, the fitting of the closures, labeling, and also handling, where a bypass may represent a possible frangible point on the container and requires special technologies which are not necessary here.

The above description discusses a number of variations and suggests a range of possible modifications which will be immediately apparent to the skilled man.

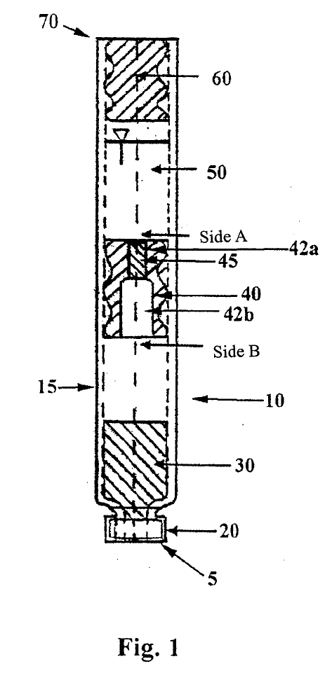

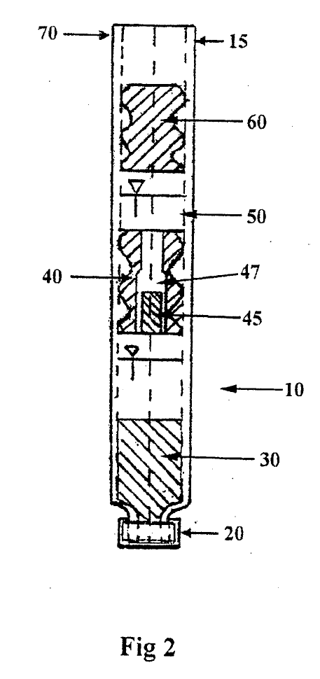

FIGS. 1 and 2 diagrammatically show the function of a double chamber container 10 according to the invention with the bypass in the separating stopper 40 closed and open. Specifically, a double chamber container 10 according to the invention is shown which is made up of a cylindrical body 15, made of glass, for example, a solid-end closure 20 at the end 5, and a liquid-end closure 60 at the end 70 of the cylindrical body 15. The lower closure 20 constitutes a rubber closure in the present case, which may be a pierceable membrane with an outer cap, which is flanged, for example, over the outer edge at the emptying end. A removable disc connected to the outer cover may be provided over this. Obviously it is also possible to provide any other closure known to the skilled man, particularly a removable closure.

The upper closure 60 may, for example, be a stopper which may be made of a suitable elastic and flexible material, such as rubber, plastics, or the like. The nature of this closure is not limited further, provided that it enables a pressure to be applied to the liquid component in the chamber 50, so that the shaped member 45 can be moved out of its position in the upper part 42a of the opening 42.

The separating stopper 40 subdivides the container 10 into two chambers 30 and 50, the chamber 30 containing a solid component such as, for example, a lyophilizate, while the other chamber 50 contains a liquid component, such as a reconstitution medium for the solid component. The separating stopper 40 has an opening 42 which exits along the longitudinal axis over the entire length of the separating stopper 40. In the upper part 42a of this opening 42 there is the shaped member 45, which seals off the two chambers 30 and 50 from one another.

If the liquid present in the chamber 50 at side A is put under pressure, the separating stopper 40 continues to adhere to the wall of the cylindrical body 15 by frictional force. If the adhesive friction of the separating stopper 40 on the wall of the cylindrical body 15 is not sufficient to the intended purpose, i.e. to prevent unwanted slipping or movement of the separating stopper 40, it may preferably be held in placed with small bumps (not shown) on the inner surface of the cylindrical body 15. The pressure at side B therefore does not increase. As a result, a differential pressure is produced between sides A and B, which causes the shaped member 45 to move in the opening 42 towards the lower part 42b in the direction of side B. As soon as the shaped member 45 frees the opening 42 with the diameter D1, the access is open and the liquid component can flow from side A through the opening 42. The opening 42 preferably contains bypass channels 47. Preferably, the opening 42 in the lower part 42b towards the solid-side end of the opening 42 (towards side B) has a larger inner diameter D2, so that the liquid component can additionally flow through the bypass channels and through the larger inner diameter of the lower part 42b of the opening 42 towards side B, as a result of which the two components are easily mixed together in a particularly rapid fashion. For example, a lyophilizate may be reconstituted.

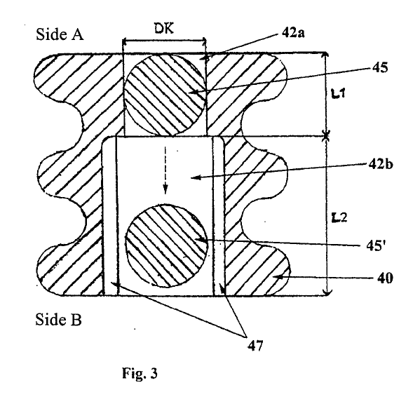

FIG. 3 shows a separating stopper 40 with the bypass open and closed, to illustrate the function of the internal bypass. In a first position the shaped member 45 is disposed in the upper part 42a (shaped member 45) and closes off the separating stopper 40 in fluid-tight manner. In a second position the shaped member 45 has been forced out of its defined first position by the manual application of force and is in the lower part 42b (shaped member 45′).

The separating stopper 40, which separates the chamber 50 with the liquid component (side A) of the two-chamber system from the chamber 30 with the solid component (side B), is preferably constructed like a conventional separating stopper at its side face which forms a seal with the wall of the cylindrical body 15.

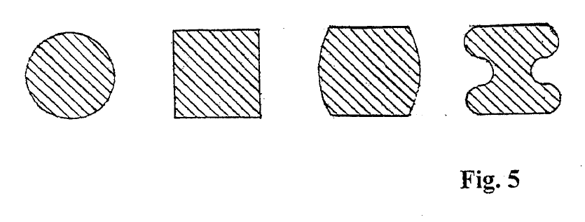

Along the longitudinal axis of the separating stopper 40 is formed the internal bypass, which in the present invention is referred to as an opening which is divided into an upper and lower part. This is achieved for example by means of an upper part 42a, starting at side A, over a length L1, which has the diameter D1 (with L1 □ D1). This upper part 42a is closed off by a shaped member 45 with a diameter DK and DK>D1, illustrated in FIG. 3 as a sphere, by way of example. However, the shaped member 45 may also be constructed as a cylinder or as a cylinder with a ball-shaped exterior or in the form of dumbbells with 2 sealing surfaces, as shown by way of example in FIG. 5.

After the length L1 the diameter of the upper part 42a widens out from D1 to the lower part 42b with a diameter D2 (with D2>D1 and L2>DK). This is particularly advantageous in the present instance, so that the frictional forces on the shaped member 45 are reduced to such an extent that in spite of the equalization of pressure between sides A and B it frees the opening 42 at the transition of D1 to D2 to such an extent that the liquid component can flow from side A to side B. To improve the transition of the liquid component from side A to side B one or more bypass channels 47 may advantageously be provided laterally on the lower part 42b with the diameter D2 and length L2. FIG. 3 shows 2 bypass channels 47 by way of example.

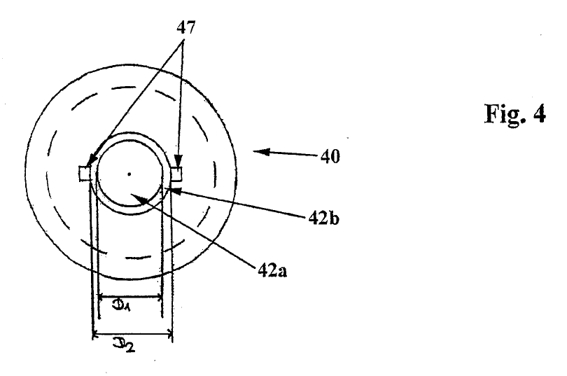

FIG. 4 shows a plan view of a preferred embodiment of a separating stopper 40 according to the invention. The upper part 42a with the diameter D1 and the lower part 42b with the larger diameter D2 are shown together with 2 bypass channels 47 which permit better access of the liquid component from the chamber 50 into the other chamber 30.

The foregoing description of the Figures serves to illustrate the apparatus according to the invention and the process according to the invention. This is intended purely as a possible procedure described by way of example without restricting the invention to its contents.

What is claimed is:

1. Double chamber container for holding and combining two separate components, at least one of which is liquid, comprising
a cylindrical body (15) with a closure (20, 60) at each of the two ends (5, 70) of the body (15), of which the closure (60) at the end of the chamber containing the liquid component (50) can be pushed by the application of pressure, and
a separating stopper (40) which can only be pushed along by the application of increased pressure, which is mounted in the cylindrical body (15), which defines the volume of the chamber (30, 50) mounted above and below as a seal between the two chambers (30, 50), with an opening (42) running right through the separating stopper along its longitudinal axis, said opening having an upper part (42a) and a lower part (42b),
wherein the upper part (42a) of the opening (42) to the side of the chamber containing the liquid component (50) is closed off in fluid-tight manner by a shaped member which is movable by the application of gentle to moderate pressure (45).

2. Double chamber container according to claim 1, characterized in that the chamber (50) contains a liquid component and the other chamber (30) contains a solid component.

3. Double chamber container according to claim 2, characterized in that the solid component is a medicament, particularly a lyophilized medicament.

4. Double chamber container according to claim 2, characterized in that the liquid component is a dissolving or dispersing medium for the solid component.

5. Double chamber container according to claim 1, characterized in that the cylindrical body (15) is made in one piece.

6. Double chamber container according to claim 1, characterized in that the separating stopper (40) is of a suitable shape, size and/or material which prevents it from being moved out of its fixed and defined position in the cylindrical body (15).

7. Double chamber container according to claim 1, characterized in that the separating stopper (40) comprises sealing lips, beads or sealing surfaces.

8. Double chamber container according to claim 1, characterized in that the shaped member (45) has a suitable three-dimensional shape such that the upper part (42a) of the opening (42) to the side of the chamber (50) is closed off.

9. Double chamber container according to claim 1, characterized in that the material of the shaped member (45) or the separating stopper (40) is selected from among natural or synthetic rubber, elastomers, thermoplastics, thermoplastic elastomers, glass, and stainless steel.

10. Double chamber container according to claim 1, characterized in that means are provided in the opening (42) which, after the shaped member (45) has left its defined first position in the upper part (42a) of the opening (42), secure it in another defined second position in the lower part (42b) of the opening (42), without preventing the access of the liquid component from the chamber (50) into the chamber (30) in this second position.

11. Double chamber container according to claim 1, characterized in that the geometric shape and/or size of the opening (42) and shaped member (45) are matched to each other such that the shaped member (45) can be pushed out of its position in the upper part (42a) by the application of pressure, but the shaped member (45) does not entirely leave the opening (42) in the lower part (42b).

12. Double chamber container according to claim 1, characterized in that the opening (42) in the upper part (42a) on the liquid component side (side A) in the chamber (50) has a different geometric shape and/or size from the opening (42) in the lower part (42b) on the solid component side (side B) in the chamber (30).

13. Double chamber container according to claim 1, characterized in that the opening (42) in the upper part (42a) on the liquid component side (side A) in the chamber (50) has a smaller inner diameter D1 than the opening (42) in the lower part (42b) on the solid component side (side B) in the chamber (30) with a larger inner diameter D2.

14. Double chamber container according to claim 1, characterized in that the opening (42) in the upper part (42a) has a diameter D1 over a length L1, where L1 is greater than or equal to D1 and the opening (42) after the L1 widens out to the diameter D2 in the lower part (42b) over a length L2, where: D2 is greater than D1 and L2 is greater than DK, where DK is the diameter of the shaped member (45).

15. Double chamber container according to claim 1, characterized in that the opening (42) in the lower part (42b) on the solid component side (side B) has one, two or more bypasses (47), particularly in the form of recesses.

16. Double chamber container according to claim 1, characterized in that the double chamber container (10) is a cylinder ampoule or double chamber carpule or the like.

17. Process for filling the double chamber container according to claim 1, comprising the steps of:
(1) inserting a separating stopper (40) which can only be moved by the application of increased pressure into the cylindrical body (15) with an opening (42) extending right through it along the longitudinal axis of the separating stopper (40), which has an upper part (42a) and a lower part (42b), the upper part (42a) of the opening (42) being sealed off in fluidtight manner by a shaped member which can be moved by slight to moderate pressure (45);
(2) packing a solid component into side B of the cylindrical body (15) provided with the separating stopper (40) or
(3) adding a liquid containing the solid component into side B of the cylindrical body (15) provided with the separating stopper (40), then lyophilising the liquid to obtain the solid component and sealing this chamber at side B,
(4) adding the liquid component to the cylindrical body (15) through the separating stopper (40) (side A) and
(5) sealing the double chamber container (10).

18. Process for mixing two separate components, at least one of which is liquid, in a double chamber container according to claim 1, comprising
a cylindrical body (15) with a closure (20, 60) at each of the two ends (5, 70) of the body (15), of which the closure (60) at the end of the chamber (50) containing the liquid component can be moved by the application of pressure, and
a separating stopper (40) which can only be moved by the application of increased pressure, arranged in the cylindrical body (15), which defines the volume of the chambers (30, 50) arranged above and below as a seal between the two chambers (30, 50), having an opening (42) running right through it along the longitudinal axis of the separating stopper, which has an upper part (42a) and a lower part (42b),
the upper part (42a) of the opening (42) at the end of the chamber containing the liquid component (50) being sealed off in fluidtight manner by a shaped member which can be moved by the application of slight to moderate pressure (45),
comprising the steps of:
applying pressure to the movable closure (60);
pressing the shaped member (45) out of its defined position into the upper part (42a) of the opening (42);
transferring the liquid component from one chamber (30) into the chamber (50) containing the solid component to obtain a mixture of both components.

19. Process according to claim 18, characterized in that the shaped member (45) after being pushed out of its defined (first) position in the upper part (42a) of the opening (42) is secured in a (second) position in the lower part (42b) of the opening (42), without interfering with the passage of the liquid component from the chamber (50) into the chamber (30) in this (second) position.
